SEQUENCE LISTING (24 sequences)

SEQ ID NO: 1 - protein, 403 aa, Dickeya zeae
ETTSSGWTLEKVVEVSRHGVRPPTEGNVKTIQQGTDRQWPTWLTHYGELTGHGYAAAVLKGHYEGQYLRQHGLLSQACPAPGDVFVWASPLQRTRETAMALMDGVFPGCGVAIQGPEDEDNDPLFHAETAGVKLDQQQVKADLQNAMQDKSAAQIQAQWQPAIDRLKQAVCLPDKPCPAFSTPWEVKENKKGNISLRGPGALANMAETIRLSYSNNNPLSEVAFGHAKTAAEVAALMPLLTANYDFTNDLPYVARRGASVLMNQIALALNTGHQADAPPDVKWLLYVAHDTNIAKLRTMLGFTWQMGDYPRGNIPPAGSLIFERWRNQQSGQRVVRIYFQAQSLDQIRGLTALDDSHPPLRSEFSMPDCQKTDIGTLCPYDAVMKRLNDAVDRTALLPVRYTP

SEQ ID NO: 2 - DNA, 1209 bp, Dickeya zeae
gaaaccacgtcgtcaggctggacgctggaaaaagtggtggaggtcagtcgccacggtgttcgtccaccgactgaaggtaatgttaaaaccattcagcaaggcaccgatcgccaatggccgacctggctgacccattacggcgaactgaccggccacggctacgcggcagcggtgttaaaagggcactacgaggggcagtacctgcgccagcatgggttactgagtcaggcttgtccggctcccggcgatgtgttcgtctgggccagtccgctacaacgcacacgggaaaccgcgatggcgctgatggacggcgtgttccccggttgtggtgtggcaattcaaggccctgaggatgaagacaacgacccgctgtttcatgccgagacggcaggcgtgaagctcgatcagcaacaggtgaaagccgacctgcaaaacgcgatgcaggacaaaagcgccgcacaaattcaggcgcaatggcaaccggctatcgatcgtctgaaacaggcggtctgcctgccggataaaccctgcccggcgttcagcaccccctgggaagtgaaagagaataaaaaagggaatatttctctgcgcggccctggggcgctggccaatatggcggagaccattcggctgtcctacagcaacaataatccactcagtgaggtggccttcggtcatgctaaaaccgccgcagaagtggcagcgctgatgccacttctgaccgccaattacgacttcaccaacgatctgccctacgtggctcgccgcggtgcgtcggtactgatgaaccagatagcgctggcgctcaacaccgggcatcaggctgatgccccaccagatgtcaaatggctgctgtatgtggcgcatgacaccaacatcgccaaactgcgtacgatgctgggctttacctggcagatgggcgactatccgcgcggcaatatcccgcccgcaggtagccttatcttcgaacgctggcgcaatcagcagtctggtcagcgtgttgtgcgtatctatttccaggcacaatcgctggatcagattcgtgggctgactgcgctggatgacagccacccgccgttacgcagcgagttctcgatgcctgactgtcagaaaaccgatatcggcaccctgtgcccgtatgacgcggtgatgaagcgccttaacgacgcggtggaccgtaccgcgctgctgccagtgcgctacacgccg

SEQ ID NO: 3 - protein, 403 aa, Dickeya dadantii
DTPSSGWTLEKVVEVSRHGVRPPTKGNVKTIQEGTDRQWPTWLTQYGELTGHGYAAAVLKGHYEGQYLRQHGLLSQACPAPGDVFVWASPLQRTRETAMALMDGVFPGCGVAVQGPEDEDNDPLFHAEAAGVKLDQQQVKADLQNAMQDKSAAQIQAQWQPAIDRLKQAVCLPDKPCPAFSTPWEVKESKKGNISLHGPEALANIAETIRLSYSNNNPLSEVAFGHAKTAAEVAALMPLLTANYDFTNDLPYMARRGASVLMNQIALALNTEHQADVPPDVKWLLYVAHDTNIAKLRTMLGFTWQMGDYPRGNIPPAGSLIFERWRNQQSGQRLVRIYFQAQSLDQIRGLAALDDSHPPLRSEFSMADCQKTDVGTLCPYDAVMKRLNDAVDRTALLPVRYTP

SEQ ID NO: 4 - DNA, 1209 bp, Dickeya dadantii
gacaccccgtcgtcaggctggacgctggaaaaagtggtggaagtcagtcgccacggtgtccgcccgccgacgaaaggcaatgttaaaaccattcaggaaggcaccgatcgccagtggccgacctggttgacgcagtacggcgagctgaccggtcacggctacgcggcagcagtgttaaaagggcactacgaggggcagtacctccgtcagcatgggttactgagtcaggcttgtccggctcccggcgacgtgttcgtctgggccagtccgctacaacgcacgcgagaaactgcaatggcgctgatggatggcgtgttccccggctgtggtgtggcggtacaaggccctgaggatgaagacaacgacccgctgtttcatgccgaggcggcgggcgtgaagctcgatcagcagcaggtgaaagccgacctgcaaaacgcgatgcaggacaaaagcgccgcgcaaattcaggcgcaatggcaaccggctatcgatcgtctgaaacaggcagtctgcctgccggataaaccctgcccggcgttcagtacgccctgggaagtgaaagagagtaaaaaagggaatatttctctgcacggccctgaggcgctggctaacatcgcggagactatccggctgtcctacagcaacaataatccgctcagcgaggtggccttcggtcatgctaaaaccgccgcagaggtggcagcgctgatgccacttctgaccgccaattacgatttcaccaacgatctgccctacatggcacgccgcggtgcgtcggtactgatgaaccagatagcgctggcgctaaacaccgagcatcaggctgatgtcccaccagatgtcaaatggctactgtatgtggcgcatgacaccaacatcgccaaactgcgtacgatgctgggctttacctggcagatgggcgactatccgcgcggcaatatcccgcccgcaggtagccttatcttcgaacgctggcgcaatcagcagtctggtcagcggctggtgcgtatctatttccaggcacaatcgctggatcagattcgtgggctagcggcgctggatgacagccacccgccgttacgcagcgagttctcgatggccgactgccagaaaaccgacgtcggcaccctgtgtccatatgacgcggtgatgaagcgccttaacgacgcggtggaccgtaccgcgctgctgccagtgcgctatacgccg

SEQ ID NO: 5 - protein, 403 aa, Dickeya zeae
ETTSSGWTLEKVVEVSRHGVRPPTEGNVKTIQQGTDRQWPSWLTHYGELTGHGYAAAVLKGHYEGQYLRQHGLLSQACPAPGDVFVWASPLQRTRETAMALVDGVFPGCGVAIQGPEDEDNDPLFHAEAAGVKLDQQQVKADLQNAMQDKSAAQIQAQWQPAIDRLKQAVCLPDKPCPAFSTPWEVKESKKGNISLHGPEVLANIAETIRLSYSNNNPLSEVAFGHAKTAAEVAALMPLLTANYDFTNDLPYVARRGASVLMNQIALALNTEHQADAPPDVKWLLYVAHDTNIAKLRTMLGFTWQMGDYPRGNIPPAGSLIFERWRNQQSGQRVVRIYFQAQSLDQIRGLTALDDSHPPLRSEFSMPDCQKTDIGTLCPYDAVMKRLNNAVDRTALLPVRYTP

SEQ ID NO: 6 - DNA, 1209 bp, Dickeya zeae
gaaaccacgtcgtcaggctggacgctggaaaaagtggtggaggtcagtcgccacggtgttcgtccgccgactgaaggcaatgttaaaaccattcagcaaggcaccgatcgccagtggccaagctggctgacccattacggcgaactgaccggccacggctacgcagcagcggtgttaaaagggcactacgaggggcagtacctgcgccagcatgggttactgagtcaggcttgtccggctcccggcgatgtgttcgtctgggccagtccgctgcaacgcacacgggaaaccgcgatggcgctagtggatggcgtgttccccggttgtggtgtggcgattcaaggccctgaggatgaagacaacgacccgctgtttcatgccgaggcggcaggcgtgaagctcgatcagcaacaggtgaaagccgacctgcaaaacgcgatgcaggacaaaagcgccgcacaaattcaggcgcaatggcaaccggctatcgatcgcctgaaacaggcggtctgtctgccggataaaccctgcccggcgttcagcacgccctgggaagtgaaagagagtaaaaaagggaatatttctctgcacggccctgaggtgctggctaacatcgcagagactatccggctgtcctacagtaacaataatccgctcagtgaggtggccttcggtcatgctaaaactgccgcagaggtggcagcactgatgccgcttctgaccgccaattacgatttcaccaacgatctgccctacgtggcacgccgcggtgcgtcggtactgatgaaccagatagcgctggcactcaacaccgagcatcaggctgatgccccaccagatgtcaaatggctgctgtatgtggcgcatgacaccaacatcgccaaactgcgtacgatgctcggctttacctggcagatgggcgactatccgcgcggcaatatcccgcccgcaggtagccttatcttcgaacgctggcgcaatcaacagtctggtcagcgcgttgtgcgtatctatttccaggcacaatcgctggatcagattcgtgggctgactgcgctggatgacagccacccgccgttacgcagcgagttctcgatgcctgactgtcagaaaaccgatatcggcaccctgtgcccgtatgacgcagtgatgaagcgccttaacaacgcggtggaccgtaccgcgctactgccagtgcgctatacgccg

SEQ ID NO: 7 - protein, 403 aa, Dickeya zeae
DTPSSGWTLEKVVEVSRHGVRPPTKGNVKTIQEGTDRQWPTWLTQYGELTGHGYAAAVLKGHYEGQYLRQHGLLSQACPAPGDVFVWASPLQRTRETAMALMDGVFPGCGVAIQGPEDEDNDPLFHAETAGVTLDQQQVKADLQNAMQDKSAAQIQAQWQPAIDRLKQAVCLPDKPCPAFSTPWEVKESKKGNISLHGPEVLANIAETIRLSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTP

SEQ ID NO: 8 - DNA, 1209 bp, Dickeya zeae
gacaccccgtcgtcaggctggacgctggaaaaagtggtggaagtcagtcgccacggtgtccgcccgccgacgaaaggcaatgttaaaaccattcaggaaggcaccgatcgccagtggccgacctggttgacgcagtacggcgagctgaccggtcacggctacgcggcagcggtgttaaaagggcactacgaggggcagtacctgcgccagcatgggttactgagtcaggcttgtccggctcccggcgatgtgttcgtctgggccagtccgctacaacgcacgcgggaaaccgcaatggcgctgatggatggcgtgttccccggttgtggtgtggcgattcaaggccctgaggatgaagacaacgacccgctgtttcatgccgagacggcaggtgtgacgctcgatcagcagcaggtgaaagccgacctgcaaaacgcgatgcaggacaaaagcgccgcgcaaattcaggcgcaatggcaacccgctatcgatcgtctgaaacaggcagtctgcctgccggataaaccctgcccggcgttcagcacgccctgggaagtgaaagagagtaaaaaagggaatatttcactgcacggccctgaggtgctggccaacatcgcggagactatccggctgtcctacagcaacaataatccgctcagcgaggtggccttcggtcatgctaaaaccgccgcagaggtggctgcgctgatgccacttctgaccgccaattacgatttcaccaacgatctgccctacgtggcacgccgcggtgcgtcggtactgatgaaccagatagcgctggcactcaacaccgagcatcaggctgatgccccaccagatgccaaatggctgctgtatgtggcgcatgacaccaacatcgccaaactgcgtacgatgctgggctttacctggcagatgggcgactatccgcgcggcaatatcccgcccgcaggtagccttatcttcgaacgctggcgcaatcagcagtctggtcagcgtgttgtgcgtatctatttccaggcacaatcgctggatcagattcgtgggctcgcggcgctggatgacagccacccgccgttacgcagcgagttctcgatgcctgactgtcagaaaaccgatatcggtaccctgtgcccgtatgacgcggtgatgaagcgccttaacgacgcggtggaccgtaccgcgctgctgccagtgcactatacgccg

SEQ ID NO: 9 - protein, 403 aa, Dickeya zeae
ETTWSGWTLEKVVEVSRHGVRPPTEGNVKTIQQGTDRQWPSWLTHYGELTGHGYAAAVLKGHYEGQYLRQHGLLSQACPAPGDVFVWASPLQRTRETAMALVDGVFPGCGVAVRGPEDEDNDPLFHAEAAGVKLDQQQVKADLQNAMQDKSAAQIQAQWQPAIDRLKQAVCLPDKPCPAFSTPWEVKENKKGNISLRGPGALANMAETIRLSYSNNNPLSEVAFGHAKTAAEVAALMPLLTANYDFTNDLPYMARRGASVLMNQIALALNTGHQADVPPDVKWLLYVAHDTNIAKLRTMLGFTWQMGDYPRGNIPPAGSLIFERWRNQQSGQRLVRIYFQAQSLDQIRGLTALDDSHPPLRSEFSMADCQKTDVGTLCPYDAVMKRLNNAVDRSALLPVHYTP

SEQ ID NO: 10 - DNA, 1209 bp, Artificial Sequence
  codon optimized Dickeya sp. phytase
gagaccacctggtccggctggaccctcgagaaggtcgtcgaggtctcccgccacggcgtccgcccccccaccgagggcaacgtcaagaccatccagcagggcaccgaccgccagtggccctcctggctcacccactacggcgagctcaccggccacggctacgccgccgccgtcctcaagggccactacgagggccagtacctccgccagcacggcctcctctcccaggcctgccccgcccccggcgacgtcttcgtctgggcctcccccctccagcgcacccgcgagaccgccatggccctcgtcgacggcgtcttccccggctgcggcgtcgccgtccgcggccccgaggacgaggacaacgaccccctcttccacgccgaggccgccggcgtcaagctcgaccagcagcaggtcaaggccgacctccagaacgccatgcaggacaagtccgccgcccagatccaggcccagtggcagcccgccatcgaccgcctcaagcaggccgtctgcctccccgacaagccctgccccgccttctccaccccctgggaggtcaaggagaacaagaagggcaacatctccctccgcggccccggcgccctcgccaacatggccgagaccatccgcctctcctactccaacaacaaccccctctccgaggtcgccttcggccacgccaagaccgccgccgaggtcgccgccctcatgcccctcctcaccgccaactacgacttcaccaacgacctcccctacatggcccgccgcggcgcctccgtcctcatgaaccagatcgccctcgccctcaacaccggccaccaggccgacgtcccccccgacgtcaagtggctcctctacgtcgcccacgacaccaacatcgccaagctccgcaccatgctcggcttcacctggcagatgggcgactacccccgcggcaacatcccccccgccggctccctcatcttcgagcgctggcgcaaccagcagtccggccagcgcctcgtccgcatctacttccaggcccagtccctcgaccagatccgcggcctcaccgccctcgacgactcccacccccccctccgctccgagttctccatggccgactgccagaagaccgacgtcggcaccctctgcccctacgacgccgtcatgaagcgcctcaacaacgccgtcgaccgctccgccctcctccccgtccactacaccccc

SEQ ID NO: 11 - protein, 404 aa, Dickeya chrysanthemi
ETTPSGWTLEKVVEVSRHGVRPPTEGNVKTIQEGTGRNWPTWLTHYGELTGHGYAAAVLKGQYEGQYLRQHGLLSQNCPTPGDVFVWASPLQRTQETAMALMDGVFPGCGVVIRGPESEDNDTLFHAETAGVQMDQEQVKASLQNAMQGKSAEQIQAEWQPAIDRLKQAVCLPDTACPAFSAKWEVKESKKGNISLHGPEALANMAETIRLSYSNNNPLGQVAFGNAGTAAAVAALMPLLTANYDFTNDLPYVARRGGSILMNQIALALNPERHQEDAPPDAKWLLYVAHDTNIAKLRAMLGFTWQMGDYPRGNIPPAGSLIFERWRNAQSGQRFVRIYFQAQSLDQIRGLTTLDNDHPPLRSEFSINGCQKIDVGTLCPYDAVMKRLNESVDRTALLPVRYAL

SEQ ID NO: 12 - DNA, 1212 bp, Dickeya chrysanthemi
  -
gaaaccaccccgtcgggctggacgctggaaaaagtcgtggaagtcagccgtcatggcgttcgcccgccgaccgaaggcaacgttaaaaccattcaggaaggcaccggccgcaactggccgacctggctgacccattacggcgaactgaccggccacggctacgcggcggccgtgctaaaaggccagtacgaagggcagtacctgcgtcagcacgggttgcttagtcagaattgcccgacgcccggcgacgtgtttgtctgggccagcccgctgcaacgcacgcaggaaaccgccatggcgctgatggatggtgtattccccggttgcggcgtggtgattcgcggcccggagagtgaagacaacgacacattgttccatgccgagacggccggcgtgcagatggatcaggaacaggtgaaagcctctttgcaaaacgcgatgcagggtaaaagcgccgaacaaattcaggcggagtggcaaccggccatcgatcgcctgaaacaggcggtctgcctgccggataccgcttgcccggcgttcagcgccaaatgggaagtgaaagaaagtaagaaagggaatatctcgctgcacggcccggaagcgctggccaatatggcggaaaccatccgcttgtcctacagcaataacaacccgctcggccaggtggccttcggcaatgccgggaccgctgccgcggtggcggcgctgatgccgctgctgaccgccaactacgatttcaccaacgacctgccctatgtggcacggcgcggcggttcgatactgatgaaccagatagccctggcgcttaacccggaacgacatcaggaggatgccccacccgatgccaaatggctgctgtacgtggcgcacgataccaacatcgcgaaactgcgtgccatgctgggcttcacctggcagatgggcgactacccgcgcggcaatatcccgcccgccggcagcctgatcttcgagcgctggcgcaacgcgcagtccggtcaacgcttcgtgcgcatctactttcaggcgcaatcgctggaccagattcgcgggctgaccacgttggataacgaccatccgccgctgcgcagcgaattctcaatcaacggttgccagaaaatcgatgtcggcaccctgtgtccgtacgatgccgtgatgaagcgcctcaacgaatcggtggatcgcaccgcgctgctgccggtgcgctacgcgctg

SEQ ID NO: 13 - protein, 403 aa, Dickeya dadantii
ETAPSGWALEKVVEISRHGVRPPTEGNVKTIQEGTGRDWPTWLTHYGELTGHGYAAAVLKGQYEGQYLRQHGLLTRNCPAPDDVFVWASPLQRTQETAMALMDGVFPGCGVVIRGPESEDNDPLFHAETAGVLVDQEQVKVDLQKAMQGKSAEQIQAEWQPAIDRLKQAVCLPDKPCPAFSAKWELKESKKGNTSLHGPEALANMAETIRLAYSNNNPLGQVAFGNARTAAAVGSLMSLLTANYDFTNDLPYVARRGGSVLMNQIALALNPEHQADAPPDAKWLLYVAHDTNIAKLRAMLGFTWQMGDYPRGNIPPAGSLIFERWRNAQSGQRFVRIYFQAQSLDQIRGLTALDNDHPPLRSEFSMNGCQKTDVGTLCPYDAVMKHLNGAVDRTALLPVRYSL

SEQ ID NO: 14 - DNA, 1209 bp, Dickeya dadantii
gaaaccgctccgtcaggctgggcactggaaaaagtggtggaaataagccgccacggcgtacgcccgccgaccgagggcaacgtcaaaaccattcaggaaggcaccgggcgcgactggccgacctggctgacccattacggcgagctgaccggtcacggttatgcggcggcggtactaaaagggcaatacgaagggcagtacctgcgccagcacggactgctgacccgcaattgcccggcgcccgacgacgtatttgtctgggccagcccgctgcaacgcacgcaggaaaccgccatggcgttgatggatggcgtatttcctggctgcggcgtcgtgattcgcggcccggagagtgaagacaacgacccgctgttccatgccgaaaccgccggcgtgctggtggatcaggaacaggtgaaggtcgacctgcaaaaagcgatgcagggcaaaagcgccgaacagattcaggcggagtggcaaccggcgatcgatcgcctgaaacaggcggtctgcctgccggataaaccctgcccggcgttcagcgccaaatgggaattgaaagagagcaaaaaagggaatacctcgctgcacggcccggaggcgctggccaacatggcggaaaccatccggctggcctacagcaacaacaacccgctcggtcaggtggcgttcggcaacgccagaacggccgccgcggtgggttcgctaatgtcgttgctgaccgccaactacgatttcaccaacgacctgccctacgtggcgcggcgcggcggttcggtactgatgaatcagatagccctggcgcttaacccggaacatcaggcggacgccccgcccgacgccaaatggctgctgtacgtggcgcacgataccaacatcgccaaattgcgcgccatgctgggtttcacctggcagatgggcgactacccgcgcggtaatatcccgcccgcgggcagcctgattttcgaacgctggcgcaacgcgcagtccggtcaacgcttcgtgcgcatctacttccaggcgcagtcgctggaccagattcgcgggctgaccgcgctggataacgaccatccgccgctgcgcagcgagttctcgatgaacggctgccagaaaaccgatgtcggcaccctgtgcccatacgatgcggtgatgaagcacctcaacggcgcggtagaccgcaccgcgctgctgccggtgcgctactcgctg

SEQ ID NO: 15 - protein, 402 aa, Dickeya paradisiaca
ASTSWSLEKVVEISRHGVRPPTEGNIKTIQEGTGREWPTWLTRYGELTGHGYAAAVLKGQYEGSYLRENALLTGACPASGEVFVWASPLQRTQETAMALMDGVFPGCGITIRGAASEEQDTLFHADDAGVTLDAEQVRADLQKAMQNKTAAQLQTGFKPDIERLQRAVCQTDNKPCPAFSAQWDVKDGKKGYPVLNGPAALANMAETIRLAYSNNAPLSQVAFGNARSAVDVGALMSLLTVNYDFTNDLPYVARRGASNVLNQIALSLSTQPQPDAPPAAKWLLFVAHDTNIAQLRTLLGFTWKQAEYPRGNIPPAGSLIFERWRNNQSGERFLRIYFQAQSLDQIRALMPLDSGNPPLRSEFTTDGCQQTEVGTLCPYDAALQRLNDAIDRTALLPVSYSR

SEQ ID NO: 16 - DNA, 1206 bp, Dickeya paradisiaca
gcctccacatcatggagcctggaaaaagtggtggaaatcagccgccacggtgtacgcccgccgacggaaggcaatatcaaaaccattcaggaaggtaccggtcgggaatggccgacctggctgacccgctatggcgagttgaccggtcacggctacgctgccgcagtgctgaaaggccagtatgaaggcagttatctgcgcgaaaatgcgctgctgaccggcgcttgcccagcctccggcgaagtatttgtctgggccagcccgttgcaacgcacgcaggaaaccgccatggcgctgatggacggtgtcttccccggctgcggcatcactatccgcggcgcggcaagcgaagaacaggacacgttgttccacgccgacgacgccggtgtcacgctggatgccgagcaggttcgcgccgatttgcaaaaagcgatgcagaacaaaaccgcagcccagcttcagaccggtttcaaaccggatatcgaacgcctgcaacgcgcggtttgccagaccgacaataaaccctgcccggcctttagcgcccaatgggacgttaaagacggcaagaaaggttatccggtgctgaatggcccggcggcgttggccaacatggcggaaaccatccgtctggcgtacagcaacaacgcgccgctcagccaggtagctttcggcaatgcccgcagtgcggtcgacgtcggcgcgctgatgtcgctgctgaccgtcaattatgatttcaccaacgatctgccctatgtcgcccgtcgcggcgcctccaatgtgctcaaccagatcgcgctctcactgtcgacacaaccgcagccggacgccccgcccgccgccaaatggctgttgttcgtggcgcacgacaccaatatcgcccagttacgcaccctgttggggttcacctggaaacaggcggaatatccgcgcggcaatattccgccggcaggcagcctgattttcgaacgttggcgcaacaatcagtctggcgagcgttttttacgcatctatttccaggcgcaatcgctggatcagatccgggcgttgatgccgctggatagcggcaacccgccgttacgcagcgaattcaccaccgatggatgtcagcaaaccgaagtggggactctgtgcccatacgacgcggccctgcaacgtcttaatgacgctatcgatcgcaccgcgctgctgccggtaagctatagtcgc

SEQ ID NO: 17 - protein, 411 aa, Escherichia coli
MQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 18 - DNA, 1296 bp, Escherichia coli
atgaaagcgatcttaatcccatttttatctcttctgattccgttaaccccgcaatctgcattcgctcagagtgagccggagctgaagctggaaagtgtggtgattgtcagtcgtcatggtgtgcgtgctccaaccaaggccacgcaactgatgcaggatgtcaccccagacgcatggccaacctggccggtaaaactgggttggctgacaccgcgcggtggtgagctaatcgcctatctcggacattaccaacgccagcgtctggtagccgacggattgctggcgaaaaagggctgcccgcagtctggtcaggtcgcgattattgctgatgtcgacgagcgtacccgtaaaacaggcgaagccttcgccgccgggctggcacctgactgtgcaataaccgtacatacccaggcagatacgtccagtcccgatccgttatttaatcctctaaaaactggcgtttgccaactggataacgcgaacgtgactgacgcgatcctcagcagggcaggagggtcaattgctgactttaccgggcatcggcaaacggcgtttcgcgaactggaacgggtgcttaattttccgcaatcaaacttgtgccttaaacgtgagaaacaggacgaaagctgttcattaacgcaggcattaccatcggaactcaaggtgagcgccgacaatgtctcattaaccggtgcggtaagcctcgcatcaatgctgacggagatatttctcctgcaacaagcacagggaatgccggagccggggtggggaaggatcaccgattcacaccagtggaacaccttgctaagtttgcataacgcgcaattttatttgctacaacgcacgccagaggttgcccgcagccgcgccaccccgttattagatttgatcaagacagcgttgacgccccatccaccgcaaaaacaggcgtatggtgtgacattacccacttcagtgctgtttatcgccggacacgatactaatctggcaaatctcggcggcgcactggagctcaactggacgcttcccggtcagccggataacacgccgccaggtggtgaactggtgtttgaacgctggcgtcggctaagcgataacagccagtggattcaggtttcgctggtcttccagactttacagcagatgcgtgataaaacgccgctgtcattaaatacgccgcccggagaggtgaaactgaccctggcaggatgtgaagagcgaaatgcgcagggcatgtgttcgttggcaggttttacgcaaatcgtgaatgaagcacgcataccggcgtgcagtttg

SEQ ID NO: 19 - protein, 412 aa, Citrobacter braakii
MEEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKADPLFNPVKMGKCSFNTLKVKNAILERAGGNIELYTQRYQSSFRTLENVLNFSQSETCKTTEKSTKCTLPEALPSELKVTPDNVSLPGAWSLSSTLTEIFLLQEAQGMPQVAWGRITGEKEWRDLLSLHNAQFDLLQRTPEVARSRATPLLDMIDTTLLTNGATENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNIQGMCSLKSFSRLIKEIRVPECAVTE

SEQ ID NO: 20 - DNA, 1236 bp, Citrobacter braakii
atggaagagcagaacggcatgaaacttgagcgggttgtgatagtgagtcgtcatggagtaagagcacctacgaagttcactccaataatgaaagatgtcacacccgatcaatggccacaatgggatgtgccgttaggatggctaacgcctcgtgggggagaacttgtttctgaattaggtcagtatcaacgtttatggttcacgagcaaaggtctgttgaataatcaaacgtgcccatctccagggcaggttgctgttattgcagacacggatcaacgcacccgtaaaacgggtgaggcgtttctggctgggttagcaccaaaatgtcaaattcaagtgcattatcagaaggatgaagaaaaagctgatcctctttttaatccggtaaaaatggggaaatgttcgtttaacacattgaaggttaaaaacgctattctggaacgggccggaggaaatattgaactgtatacccaacgctatcaatcttcatttcggaccctggaaaatgttttaaatttctcacaatcggagacatgtaagactacagaaaagtctacgaaatgcacattaccagaggctttaccgtctgaacttaaggtaacgcctgacaatgtatcattacctggtgcctggagcctttcttccacgctgactgagatatttctgttgcaagaggcccagggaatgccacaggtagcctgggggcgtattacgggagaaaaagaatggagagatttgttaagtctgcataacgctcagtttgatcttttgcaaagaactccagaagttgcccgtagtagagccacaccattactcgatatgatagacactacactattgacaaatggtgcaacagaaaacaggtatggcataaaattacctgtatctctgttgtttattgctggtcatgataccaatcttgcaaatttaagcggggctttagatcttaactggtcgctacccggtcaacccgataatacccctcctggtggggagcttgtattcgaaaagtggaaaagaaccagtgataatacggattgggttcaggtttcatttgtttatcagacgctgagagatatgagggatatacaaccgttgtcgttagaaaaacctgccggcaaagttgatttaaaattaattgcatgtgaagagaaaaatattcagggaatgtgttcgttaaaaagtttttccaggctcattaaggaaattcgcgtgccagagtgtgcagttacggaa

SEQ ID NO: 21 - protein, 446 aa, Buttiauxella sp.
MTISAFNRKKLTLHPGLFVALSAIFSLGSTAYANDTPASGYQVEKVVILSRHGVRAPTKMTQTMRDVTPNTWPEWPVKLGYITPRGEHLISLMGGFYRQKFQQQGILSQGSCPTPNSIYVWADVDQRTLKTGEAFLAGLAPECHLTIHHQQDIKKADPLFHPVKAGTCSMDKTQVQQAVEKEAQTPIDNLNQHYIPFLALMNTTLNFSTSAWCQKHSADKSCDLGLSMPSKLSIKDNGNKVALDGAIGLSSTLAEIFLLEYAQGMPQAAWGNIHSEQEWASLLKLHNVQFDLMARTPYIARHNGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 22 - DNA, 1338 bp, Buttiauxella sp.
atgacgatctctgcgtttaaccgcaaaaaactgacgcttcaccctggtctgttcgtagcactgagcgccatattttcattaggctctacggcctatgccaacgacactcccgcttcaggctaccaggttgagaaagtggtaatactcagccgccacggggtgcgagcaccaaccaaaatgacacagaccatgcgcgacgtaacacctaatacctggcccgaatggccagtaaaattgggttatatcacgccacgcggtgagcatctgattagcctgatgggcgggttttatcgccagaagtttcaacaacagggcattttatcgcagggcagttgccccacaccaaactcaatttatgtctgggcagacgttgatcagcgcacgcttaaaactggcgaagctttcctggcagggcttgctccggaatgtcatttaactattcaccaccagcaggacatcaaaaaagccgatccgctgttccatccggtgaaagcgggcacctgttcaatggataaaactcaggtccaacaggccgttgaaaaagaagctcaaacccccattgataatctgaatcagcactatattccctttctggccttgatgaatacgaccctcaacttttcgacgtcggcctggtgtcagaaacacagcgcggataaaagctgtgatttagggctatccatgccgagcaagctgtcgataaaagataatggcaacaaagtcgctctcgacggggccattggcctttcgtctacgcttgctgaaattttcctgctggaatatgcgcaagggatgccgcaagcggcgtgggggaatattcattcagagcaagagtgggcgtcgctactgaaactgcataacgtccagtttgatttgatggcacgcacgccttatatcgccagacataacggcacgcctttattgcaggccatcagcaacgcgctgaacccgaatgccaccgaaagcaaactgcctgatatctcacctgacaataagatcctgtttattgccggacacgataccaatattgccaatatcgcaggcatgctcaacatgcgctggacgctacctgggcaacccgataacacccctccgggcggcgctttagtctttgagcgtttggccgataagtcagggaaacaatatgttagcgtgagcatggtgtatcagactctcgagcagttgcgctcccaaacaccacttagccttaatcaacctgcgggaagcgtacagctaaaaattcctggctgtaacgatcagacggctgaaggatactgcccgctgtcgacgttcactcgcgtggttagccaaagcgtggaaccaggctgccagctacag

SEQ ID NO: 23 - protein, 467 aa, Aspergillus niger
MGVSAVLLPLYLLSGVTSGLAVPASRNQSSCDTVDQGYQCFSETSHLWGQYAPFFSLANESVISPEVPAGCRVTFAQVLSRHGARYPTDSKGKKYSALIEEIQQNATTFDGKYAFLKTYNYSLGADDLTPFGEQELVNSGIKFYQRYESLTRNIVPFIRSSGSSRVIASGKKFIEGFQSTKLKDPRAQPGQSSPKIDVVISEASSSNNTLDPGTCTVFEDSELADTVEANFTATFVPSIRQRLENDLSGVTLTDTEVTYLMDMCSFDTISTSTVDTKLSPFCDLFTHDEWINYDYLQSLKKYYGHGAGNPLGPTQGVGYANELIARLTHSPVHDDTSSNHTLDSSPATFPLNSTLYADFSHDNGIISILFALGLYNGTKPLSTTTVENITQTDGFSSAWTVPFASRLYVEMMQCQAEQEPLVRVLVNDRVVPLHGCPVDALGRCTRDSFVRGLSFARSGGDWAECFA

SEQ ID NO: 24 - DNA, 1401 bp, Aspergillus niger
atgggcgtctctgctgttctacttcctttgtatctcctgtctggagtcacctccggactggcagtccccgcctcgagaaatcaatccagttgcgatacggtcgatcaggggtatcaatgcttctccgagacttcgcatctttggggtcaatacgcaccgttcttctctctggcaaacgaatcggtcatctcccctgaggtgcccgccggatgcagagtcactttcgctcaggtcctctcccgtcatggagcgcggtatccgaccgactccaagggcaagaaatactccgctctcattgaggagatccagcagaacgcgaccacctttgacggaaaatatgccttcctgaagacatacaactacagcttgggtgcagatgacctgactcccttcggagaacaggagctagtcaactccggcatcaagttctaccagcggtacgaatcgctcacaaggaacatcgttccattcatccgatcctctggctccagccgcgtgatcgcctccggcaagaaattcatcgagggcttccagagcaccaagctgaaggatcctcgtgcccagcccggccaatcgtcgcccaagatcgacgtggtcatttccgaggccagctcatccaacaacactctcgacccaggcacctgcactgtcttcgaagacagcgaattggccgataccgtcgaagccaatttcaccgccacgttcgtcccctccattcgtcaacgtctggagaacgacctgtccggtgtgactctcacagacacagaagtgacctacctcatggacatgtgctccttcgacaccatctccaccagcaccgtcgacaccaagctgtcccccttctgtgacctgttcacccatgacgaatggatcaactacgactacctccagtccttgaaaaagtattacggccatggtgcaggtaacccgctcggcccgacccagggcgtcggctacgctaacgagctcatcgcccgtctgacccactcgcctgtccacgatgacaccagttccaaccacactttggactcgagcccggctacctttccgctcaactctactctctacgcggacttttcgcatgacaacggcatcatctccattctctttgctttaggtctgtacaacggcactaagccgctatctaccacgaccgtggagaatatcacccagacagatggattctcgtctgcttggacggttccgtttgcttcgcgtttgtacgtcgagatgatgcagtgtcaggcggagcaggagccgctggtccgtgtcttggttaatgatcgcgttgtcccgctgcatgggtgtccggttgatgctttggggagatgtacccgggatagctttgtgagggggttgagctttgctagatctgggggtgattgggcggagtgttttgct

(19) United States
(12) Patent Application Publication
(10) Pub. No.: US 2020/0214319 A1
(43) Pub. Date: Jul. 9, 2020

(54) Use of Bacterial 3-Phytase for Feed or Food Products

(71) Applicant: Clariant International Ltd., Muttenz (CH)

(72) Inventors: Thomas EISELE, Eichenau (DE); Michael HOESL, München (DE); Michael KRAUS, München (DE); Christoph LEICHSENRING, München (DE); Felix LIST, München (DE); Lutz ROECHER, München (DE); Frank WALLRAPP, Gauting (DE); Helge JOCHENS, Baierbrunn (DE)

(21) Appl. No.: 16/310,268

(22) PCT Filed: Jun. 14, 2017
(86) PCT No.: PCT/EP2017/064560

§ 371 Date: Dec. 14, 2018

(30) Foreign Application Priority Data
Jun. 16, 2016 (EP) 16174759.7

(51) Int. Cl. A23K 20/189; A23K 20/158; A23K 20/163; A23K 20/174; A23K 10/18; A23K 20/20; A23K 20/179; C12N 9/16

(52) U.S. Cl. CPC A23K 20/189; A23K 20/158; A23K 20/163; A23K 20/174; C12Y 301/03008; A23K 20/20; A23K 20/179; C12N 9/16; A23K 10/18

(57) ABSTRACT

The present invention relates to the use of bacterial 3-phytases for the production of feed or food products. Further, the present invention relates to the use of bacterial 3-phytases for feeding animals and for the production of a food or feed additive.

### BACKGROUND

Phytases are enzymes which can remove phosphate from organophosphate compounds such as phytate. Many types of phytases are known in the art and some of them are already used as an additive to animal feed products in order to improve metabolization and the utilization of phytate naturally present within animal feed stuff such as corn, wheat and soybeans. The classification of the phytases depends on the starting position of cleavage of phosphate from the phytate molecule in the first place. 3-phytases start with the phosphate on position 3 of the phytate molecule. 6-phytases start with the phosphate on position 6. Depending on the type of phytase, further cleavage of other positions occurs.

The addition of the phytases to the animals' feed enables better metabolization of the natural phytate content of the feed, as phosphate is an important element of an animal's diet, but also leads to a decrease of remaining phosphor in the animals' excrements. High phosphorous contents in animals' excrements usually leads to severe limitations of its use as manure as many countries set strict limitations.

In order to overcome these drawbacks of an insufficient natural phytate metabolization and a costly artificial phosphorous enrichment of feed which is also known to particularly increase remaining phosphorous content in manure, phytases have already been introduced in animal feed products to increase phytate metabolization in the animal's stomach and digestive tract.

Examples for such phytases which are currently commercially available originate from Escherichia coli, Citrobacter braakii, Buttiauxella species and Aspergillus niger.

Each of these phytase products has limitations in at least specific activity, dosage requirements, reaction time and accumulation of inhibiting intermediate compounds. First of its kind products contained fungal 3-phytases which require high enzyme dosing in order to achieve the desired effect. Currently available, commercial feed products try to overcome this effect by using 6-phytases of bacterial origin. A severe drawback of these 6-phytases is, however, an accumulation of the intermediate compound inositol-tetraphosphate which severly limits efficient phytate metabolization. In addition phytate and its intermediates, particularly inositol-tetraphosphate, form complexes with divalent ions such as Ca²⁺, Zn²⁺ and Mg²⁺. Further, many of the phytase products known within the art require the use of a high amount of phytase to guarantee a sufficient phosphate release regarding the actual critical retention time of the feed in the animals stomach of a maximum of 1.5 hours.

Therefore, there is a need within the state of the art to provide higher efficient ways to improve phytate metabolization when feeding animals. The inventors of the present invention have now surprisingly found that this need can be remedied by using bacterial 3-phytases.

### SUMMARY

The present invention relates to the use of bacterial 3-phytases for the production of feed or food products. Further, the present invention relates to the use of bacterial 3-phytases for feeding animals and for the production of a food or feed additive. Within a first aspect, the present invention is therefore directed to the use of at least one bacterial 3-phytase for the production of a feed or food product. Within a second aspect, the present invention is directed to the use of at least one bacterial 3-phytase for feeding animals. And in a final aspect the present aspect is directed to the use of at least one bacterial 3-phytase for the production of a food or feed additive.

Within the present application the term 3-phytase “is to be understood as any phytase falling in the class of E.C. 3.1.3.8 and are also referred to as: 1-phytase; myo-inositol-hexakisphosphate 3-phosphohydrolase; phytate 1-phosphatase or phytate 3-phosphatase.

The term “bacterial phytase” within the present application is to be understood as comprising any phytase which is of bacterial origin. “Bacterial origin” pertains to any phytase of modified or non-modified form such as so-called wildtype-phytases isolated from any kind of bacterial genome but also to phytases in modified such as genetically modified or genetically engineered or mutated form.

According to the present invention phytases from the genus Dickeya and Serratia are particularly preferred, wherein phytases from Dickeya sp., Serratia sp., Dickeya zeae, Dickeya chrysanthemi, Dickeya dadantii, Dickeya solani or Dickeya paradisiaca are most preferred.

Within a particularly preferred embodiment phytases are used which are characterized by less than 25% (wt./wt. free phosphate) accumulation of the intermediate product inositol-tetraphosphate after 35% inorganic phosphate release within an assay of 2.7 mmol/L phytate at 37° C. and pH 5.5 using an enzyme dosage of 0.2 U/mL. Such phytases are particularly advantageous as an accumulation of the intermediate product inositol-tetraphosphate hampers full phosphate release of the phytate within the relevant residual time of the feed within the animals' stomach. Further, accumulation of the intermediate inositol-tetraphosphate hampers divalent ion utilization. Phytases commonly used for animal feed products accumulate huge amounts of this intermediate product prohibiting full phosphor release before the feed mass is leaving the animals' stomach for further digestion.

Less than 25% (wt./wt.) accumulation of the intermediate product inositol-tetraphosphate is further achieved by implementing an extremely low enzyme dosage of 0.2 U/mL. Such phytases are therefore also highly advantageous for economical reasons. Particularly preferred is the use of phytases characterized by less than 20% (wt./wt. free phosphate) accumulation or by less than 15% (wt./wt. free phosphate) accumulation and most preferred of less than 10% (wt./wt. free phosphate) accumulation of the intermediate product inositol-tetraphosphate after 35% inorganic phosphate release within an assay of 2.7 mmol/L phytate at 37° C. and pH 5.5 using an enzyme dosage of 0.2 U/mL.

Within a further embodiment, phytases are used which are characterized by a phosphate release of at least 15% from 2.7 mmol/L phytate at pH 5.5 after 1 hour using an enzyme concentration of 0.21 μg/mL. The use of these phytases is preferred because retention time of the feed within the stomach of the animal is usually around 1 hour. Therefore, phytases are preferred which release a majority of phosphor within the timeframe of 1 hour. The more phosphate can be released within the stomach retention time the less phytase can be used within the feed product. This will further contribute to economical advantages. It is thereby particularly preferred to use phytases characterized by a phosphate release of at least 20%, even more preferred by at least 25% also particularly preferred by at least 30%, or by at least 35% as well as by at least 40% or at least 45% and most preferred by at least 50% from 2.7 mmol/L phytate at pH 5.5 after 1 hour using an enzyme concentration of 0.21 μg/mL.

As temperature during digestion in the animal's stomach is greatly varying phytases with a temperature optimum of from 37 to 42° C. are used within a particularly preferred embodiment, wherein temperature optimum of from 35 to 45° C. are particularly preferred and from 30 to 50° C. are most preferred. In case the phytases of the present invention are used to feed fish, phytases with a temperature optimum of from 1 to 45° C., preferably from 15 to 35° C. are used.

As pH within the animals' stomach is greatly varying due to the respective feed ratio consumed at a time within a particularly preferred embodiment phytases are used which show a pH optimum within a range of from pH 3.5 to 4.5, more preferred from pH 3.0 to 5.0, and most preferred from pH 2.5 to 5.5. Another particularly preferred range is from pH 5.0 to 5.5.

Within a further particularly preferred embodiment, bacterial 3-phytases with a sequence identity of at least 70% to SEQ ID NO 1, preferably at least 75% identity to SEQ ID NO 1, further preferred of at least 80% identity to SEQ ID NO 1, particularly preferred of at least 85% identity to SEQ ID NO1 also preferred of at least 90% SEQ ID NO1, moreover particularly preferred of at least 95% to SEQ ID NO 1 and most preferred of at least 99% sequence identity to SEQ ID NO 1 are used. Particularly preferred bacterial 3-phytases are SEQ ID NO 3, SEQ ID NO 5, SEQ ID NO 7, SEQ ID NO 9, SEQ ID NO 11, SEQ ID NO 13 and SEQ ID NO 15.

The term “feed” product as used within the present application pertains to any product known to a person skilled in the art as suitable for feeding any kind of animal, preferably mammals and birds, such as but not limited to ruminants, pigs, deer, poultry but also comprises fish or other aquatic livestock generally referred to as seafood. Particularly preferred are feed products for non-ruminant animals, e.g. poultry, broilers, birds, chickens, turkeys, ducks, geese, and fowl; ruminant animals e.g. cows, cattle, horses, and sheep; pigs, swine, piglets, growing pigs, and sows; companion animals including but not limited to: cats, dogs, rodents, and rabbits; fish including but not limited to salmon, trout, tilapia, catfish and carp; and crustaceans including but not limited to shrimp and prawn.

The term “food” product as used within the present application pertains to any product known to a person skilled in the art as suitable human consumption.

The feed or food product of the present invention preferably contains starch. The starch-containing feed components typically include vegetable material such as cereal(s), e.g., one or more of corn (maize), wheat, barley, rye, rice, sorghum and millet, and/or tubers such as potatoes, cassava and sweet potato. The vegetable material may be milled, e.g., wet or dry milled grain, or distillers dry grain solids.

The feed or food product of the present invention preferably contains protein-rich feed ingredients such as soybean (preferably soybean meal), rapeseed, palm kernel, cotton seed and sunflower.

Within a further embodiment, the feed or food product comprises at least one compound selected from the group consisting of vitamins, minerals, organic acids, probiotic components, oils, fats, pigments, growth factors and antimicrobial agents.

Within a further embodiment, the feed or food product comprises at least one one or more enzymes in addition to the bacterial 3-phytase, particularly feed enzymes which improve the digestibility of the feed, e.g. another phytase, an amylase or a protease, aminopeptidase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, pepti-doglutaminase, peroxidase, polyphenoloxidase, proteolytic enzyme, ribonuclease, transglutaminase, or xylanase. The feed enzyme(s) may be derived from microorganisms such as bacteria or fungi or from plants or animals.

Within another preferred embodiment the feed or food product is in granulate, compactate, extrudate or liquid form.

Another aspect of the present invention pertains to the use of bacterial 3-phytases for feeding animals. Thus, the bacterial 3-phytases are not incorporated into a separate product but directly given to the animals.

If the bacterial 3-phytase is directly given to the animal, the phytase is preferably provided in the form of an extrudate or granulate, particularly a coated granulate, e.g. with a coating comprising a salt, e.g. at least 60% w/w of the salt. The salt may be selected from the group consisting of NaCl, KCl, Na₂SO₄, K₂SO₄ and MgSO₄.

Another aspect of the present invention pertains to the use of bacterial 3-phytases for the production of a food or feed additive. A food or feed additive is a product which can be incorporated into a commercial food product such as pelleted or extrudated mixtures of grains, corn, hay, molasses and straw and may be in dry or liquid form. 3-phytases as defined before for use for the production of a feed or food product are particularly preferred.

As used herein “liquid form” pertains to a composition preferably also comprising a buffer, a stabilizer, and an anti-microbial agent and may even comprise at least one further enzyme, wherein the enzyme is an amylase, a cellulase, a lactase, a lipase, a protease, a catalase, a xylanase, a beta-glucanase, a mannanase, an amylase, an amidase, an epoxide hydrolase, an esterase, a phospholipase, a transaminase, an amine oxidase, a cellobiohydrolase, an ammonia lyase, or any combination thereof.

As used herein “dry form” pertains to a composition preferably also comprising one or more of the following components: a carrier, a buffer, a stabilizer, a binding agent, a plasticizer, an anti-microbial agent. Particularly preferred are dry forms comprising sucrose, sodium chloride, sorbitol, sodium citrate, potassium sorbate, sodium benzoate, sodium propionate, guar gum and/or wheat flour.

### BRIEF DESCRIPTION OF THE DRAWINGS

The present invention is in the following further described by examples and figures. It is emphasized that the examples and figures have only exemplary character and do not limit the scope of the present invention.

FIG. 1 shows a comparison between bacterial 3-phytases according to the present invention and bacterial 6-phytases currently used within the state of the art for feeding animals with respect to accumulation of the intermediate product inositol-tetraphosphate at pH 5.5 and 37° C.

FIG. 2 shows a comparison of bacterial 3-phytases according to the present invention and fungal 3-phytases which are also known within the state of the art to be used for feeding animals and have been used in the past relating to a phosphate release pertaining to the same amount of phytase used within the assay at pH 5.5 and 37° C.

FIG. 3 shows a comparison of bacterial 3-phytases according to the present invention and bacterial 6-phytases currently used within the state of the art for feeding animals with respect to phosphate release from the intermediate product inositol-tetraphosphate at pH 5.5 and 37° C.

FIG. 4 shows a comparison between bacterial 3-phytases according to the present invention and bacterial 6-phytases currently used within the state of the art for feeding animals with respect to accumulation of the intermediate product inositol-tetraphosphate at pH 5.0 and 40° C.

FIG. 5 shows a comparison of bacterial 3-phytases according to the present invention and fungal 3-phytases which are also known within the state of the art to be used for feeding animals and have been used in the past relating to a phosphate release pertaining to the same amount of phytase used within the assay at pH 5.0 and 40° C.

FIG. 6 shows a comparison of bacterial 3-phytases according to the present invention and bacterial 6-phytases currently used within the state of the art for feeding animals with respect to phosphate release from the intermediate product inositol-tetraphosphate at pH 5.0 and 40° C.

### DETAILED DESCRIPTION

### Methods

The following methods have been applied within the examples:

### State of the Art Phytases

The following phytases have been tested within the examples:

| Short name | Species | SEQ ID NO |
|---|---|---|
| E. coli | Escherichia coli | 17 |
| Cit. braaki | Citrobacter braakii | 19 |
| Butt. sp | Buttiauxella sp. | 21 |
| A. niger | Aspergillus niger | 23 |

### Determination of Enzyme Concentrations

SEQ ID NO 1 concentration was determined via UV absorbance using its molar extinction coefficient. All other phytase solutions and extracts were quantified via an in house SDS gel quantification method using a SEQ ID NO 1 calibration curve. Phytase samples were applied to a SDS gel which was subsequently stained with Sypro Ruby (Thermo Fisher: S12000). The gel image was recorded on a standard Bio-Rad gel documentation instrument. Image analysis was performed using ImageLab software (Bio-Rad). Protein concentration was determined by signal integration of the phytase specific SDS gel bands using a SEQ ID NO 1 calibration curve on the same SDS gel.

### Determination of Phytase Activity

5 μL enzyme solution (in 100 mmol/L sodium acetate buffer, pH 5.5, 0.05% (w/v) Triton X-100) were incubated with 95 μL 2.88 mmol/L sodium phytate (Sigma 68388, lot BCBM4006V) in 100 mmol/L sodium acetate buffer, pH 5.5 at 37° C. for 15 min. The reaction was stopped by adding 100 μL 10% (w/v) trichloroacetic acid. Subsequently, 100 μL of the stopped enzymatic reaction was mixed with 100 μL molybdate reagent (aqueous solution of 1.2% (w/v) ammonium molybdate; 4.4% (v/v) sulfuric acid, and 27 mg/mL (w/v) ferrous sulfate) and the solution was incubated for 15 min at room temperature. Absorbance at 700 nm was determined and the amount of released inorganic phosphate was calculated using inorganic phosphate standard solutions (0-1.5 mmol/L). One unit of phytase activity was defined as the amount of enzyme that releases 1 μmol phosphate per min at 37° C.

### Determination of Phytate Content in the Commercial Phytate Preparation

The standard substance phytic acid sodium salt hydrate (Sigma Art. 68388, lot BCBM4006V) contains phytate, sodium and water. The phytate content is not available by a certificate of analysis and was determined by acid hydrolysis and subsequent determination of free phosphorus by ICP-OES. 200 mg phytate sodium salt hydrate (Sigma Art. 68388, lot BCBM4006V) was weighted in a teflon tube. Subsequently, 1 mL water, 1 mL sulfuric acid 98%, and 4 mL hydrogen peroxide solution 30% were added. The tube was closed with the lid and bursting cap and incubated at 300° C. for 1 h in an autoclave. The hydrolysate was transferred quantitatively to a 50 mL volumetric flask and fill up to 50 mL with water. This solution was analysed for the content of phosphorus by ICP-OES according DIN EN ISO 11885 E22. The content of phytate was calculated by the weight, the content of phosphorus and molar masses. The content of free phytate in phytic acid sodium salt hydrate (Sigma Art. 68388, lot BCBM4006V) was 54.7%.

### Determination of Different Inositol-Phosphate Species by HPAEC-UV

The determination of the different inositol-phosphate species was performed by high performance anion exchange chromatography with post column derivatisation using an UV detector at 290 nm (HPAEC-UV). The chromatographic system was a Dionex ICS-3000 with dual pump (IC-3000 DP), dual column oven (ICS-3000 DC), cooled autosampler (ICS-3000 AS) and UV detector (ThermoFisher MWD-3000). Instead of a post column delivery system a knitted reaction coil (4 mm system, 375 μL) was used for mixing the flows of both pump systems to perform the derivatisation reaction before detection. System 1 was used to separate the different inositol-phosphate species by using a Dionex CarboPac PA100 Guard column 4×50 mm and a Dionex CarboPac PA100 analytical column 4×250 mm. For gradient elution, HPLC grade water (pump system 1/canal A) and 0.5 M hydrochloric acid (pump system 1/canal B) were used at a flow rate of 1.0 mL/min. The gradient conditions were as follows: 0 min. 5% B; 0-8 min. 5-10% B; 8-25 min. 10-35% B; 25-35 min. 35-100% B; 35-42 min. 100% B; 42-43 min 100-5% B; 43-55 min 5% B. Pump system 2 provides the post column reaction solution (0.33 M perchloric acid with 0.1% Fe(NO₃)₃) to a tee connector in combination with the knitting coil at a flow rate of 0.4 mL/min. The injection volume was about 50 μL at a column temperature of 30° C. and an autosampler temperature of 10° C. The run time was 55 min. The signals were detected at 290 nm and manually intregrated. The standard stock solution was prepared as follows: 100 mg phytic acid sodium salt hydrate (Sigma Art. 68388, lot BCBM4006V) was diluted in 100 mL Phosphate Standard Solution 1000 mg/L (Merck Art. 119898). The dilutions to several calibration levels were performed with water (HPLC grade) to end up with the following concentrations: 0.1 mg/mL Phytic acid sodium salt hydrate/Phosphate, 0.2 mg/mL Phytic acid sodium salt hydrate/Phosphate, 0.4 mg/mL Phytic acid sodium salt hydrate/Phosphate, 0.6 mg/mL Phytic acid sodium salt hydrate/Phosphate, and 0.8 mg/mL Phytic acid sodium salt hydrate/Phosphate.

### List of Retention Times of the Inositol-Phosphate Species:

| Substance | Retention time [min] |
|---|---|
| Phsophate | 3.45 |
| 1,4-Inositol-diphosphate | 11.81 |
| 4,5-Inositol-diphosphate | 12.33 |
| 1,2,3-Inositol-triphosphate | 17.86 |
| 1,4,5-Inositol-triphosphate | 18.27 |
| 1,5,6-Inositol-triphosphate | 18.69 |
| 1,2,4,5-Inositol-tetraphosphate | 24.38 |
| 1,3,4,5-Inositol-tetraphosphate | 25.39 |
| 2,4,5,6-Inositol-tetraphosphate | 27.63 |
| 1,4,5,6-Inositol-tetraphosphate | 28.82 |
| 1,2,3,4,5-Inositol-pentaphosphate | 32.12 |
| 1,2,4,5,6-Inositol-pentaphosphate | 34.40 |
| 1,2,3,4,5,6-Inositol-hexaphosphate (Phytate) | 39.07 |

### EXAMPLES

### Example 1 Determination of Phytate Degradation Pattern

Enzymatic reactions were performed by incubating a 2.7 mmol/L phytate solution (Sigma 68388, lot BCBM4006V) in 100 mmol/L sodium acetate buffer, pH 5.5 at 37° C. with 0.2 U/mL of phytase. Time resolved enzymatic phytate degradation data was recorded by stopping the enzymatic reaction at different time points via immediate incubation at 99° C. for 10 min in a thermo shaker. The samples were analyzed according to method “Determination of different inositol-phosphate species by HPAEC-UV”. Peak areas of inositol-phosphate isomers (inositol-phosphates with same amount of phosphate residues) were summed up. Subsequently, peak areas were corrected by normalization of the signals to the amount of phosphate residues of the different inositol-phosphate species (i.e. phytate contains six phosphate residues resulting in a six times higher detector response factor than the one of inorganic phosphate). For comparison of the degradation pattern, data of kinetic time points were taken at which equal amounts of phosphate had been released.

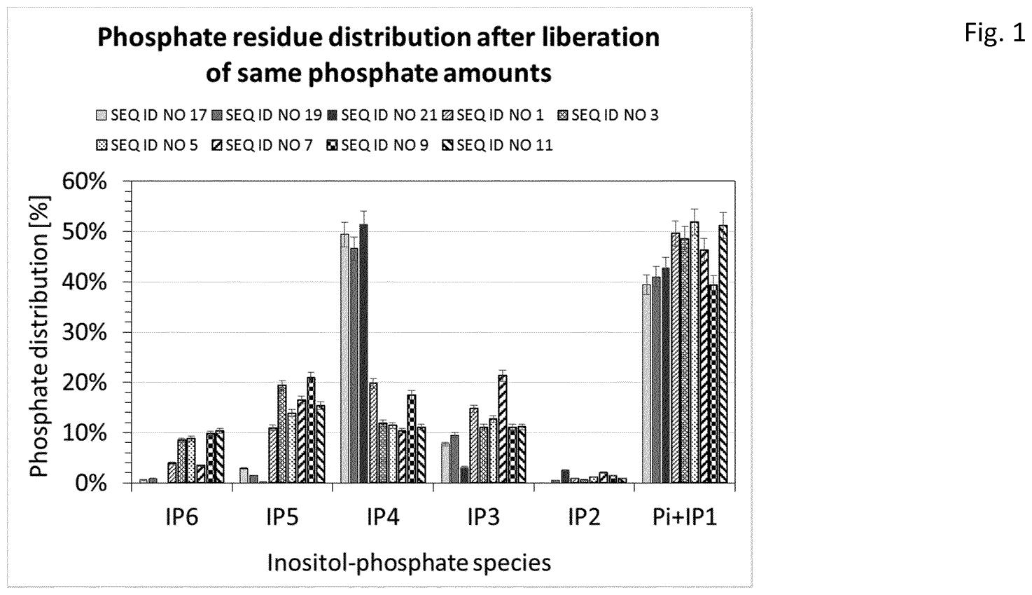

The results are shown in FIG. 1.

### Example 2 Determination of Phosphate Release Data at Same Enzyme Amounts of Phytase

5 μL of enzyme solution (in 100 mmol/L sodium acetate buffer, pH 5.5, 0.05% (w/v) Triton X-100) are incubated with 95 μL 2.88 mmol/L sodium phytate (Sigma 68388, lot BCBM4006V) in 100 mmol/L sodium acetate buffer, pH 5.5 at 37° C. for 1 h. The final enzyme concentration in the reaction is 0.21 μg/mL. The reaction is stopped by adding 100 μL 10% (w/v) trichloroacetic acid. Subsequently, the stopped enzymatic reaction is diluted 1/10 and 100 μL of the dilution is mixed with 100 μL molybdate reagent (aqueous solution of 1.2% (w/v) ammonium molybdate; 4.4% (v/v) sulfuric acid, and 27 mg/mL (w/v) ferrous sulfate). The solution is incubated for 15 min at room temperature. Absorbance at 700 nm is determined and the amount of released inorganic phosphate is calculated using inorganic phosphate standard solutions (0-1.5 mmol/L).

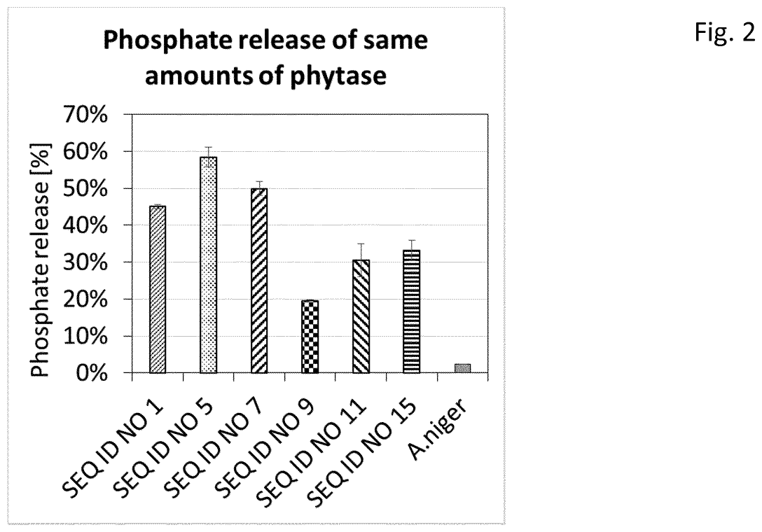

The results are shown in FIG. 2.

### Example 3 Determination of Phosphate Release from Inositol-Tetraphosphate

An Inositol-tetraphosphate preparation was produced by incubation of 2.7 mmol/L phytate in 100 mmol/L sodium acetate buffer, pH 5.5 at 37° C. for 1 h with 0.1 U/mL of SEQ ID NO 17.

After the reaction, the enzyme was inactived by incubation at 95° C. for 30 min. The resulting 2.7 mmol/L inositol-tetraphosphate solution was used as a substrate in an enzymatic reaction according to Example 2 with a final phytase concentration of 0.2 U/mL.

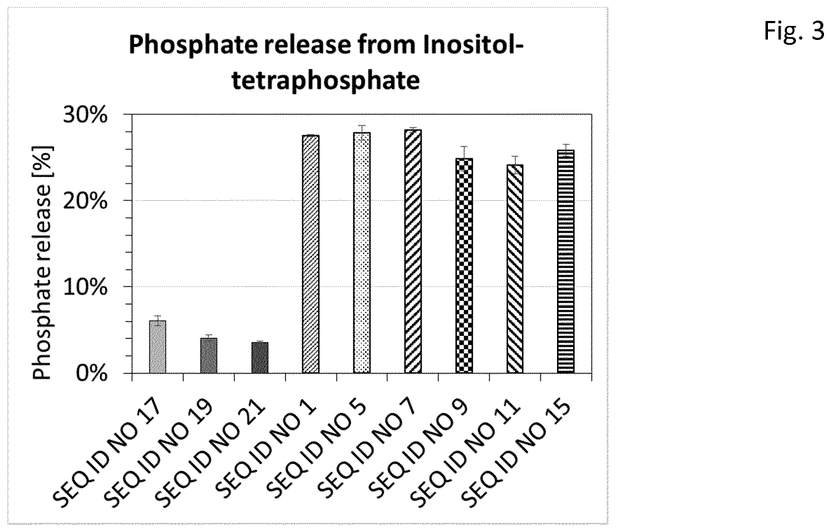

The results are shown in FIG. 3. The abbreviations used in the figure are: IP6: Inositol-hexaphosphate; IP5: Inositol-pentaphosphate; IP4: Inositol-tetraphosphate; IP3: Inositol-triphosphate; IP2: Inositol-diphosphate; IP1: Inositol-monophosphate; Pi: phosphate.

### Example 4 Determination of Phytate Degradation Pattern

Example 4 has been conducted as example 1, however, pH was set to 5 and incubation temperature was 40° C.

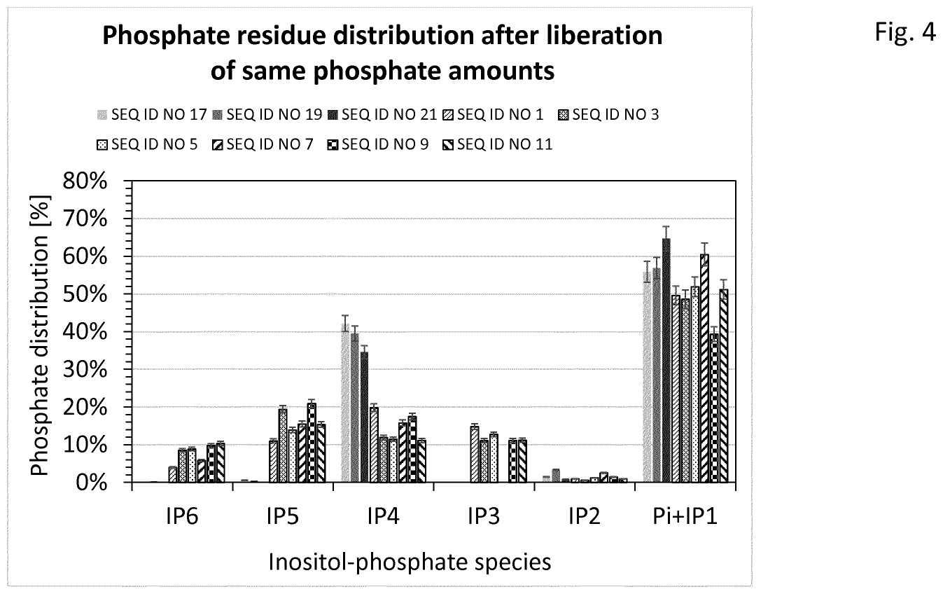

Results are shown in FIG. 4.

### Example 5 Determination of Phytate Degradation Pattern

Example 2 has been conducted as example 1, however, pH was set to 5 and incubation temperature was 40° C.

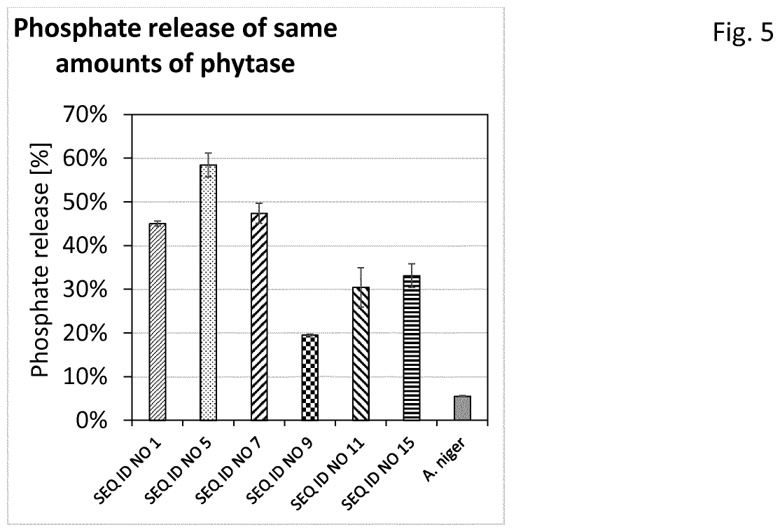

Results are shown in FIG. 5.

### Example 6 Determination of Phytate Degradation Pattern

Example 3 has been conducted as example 1, however, pH was set to 5 and incubation temperature was 40° C.

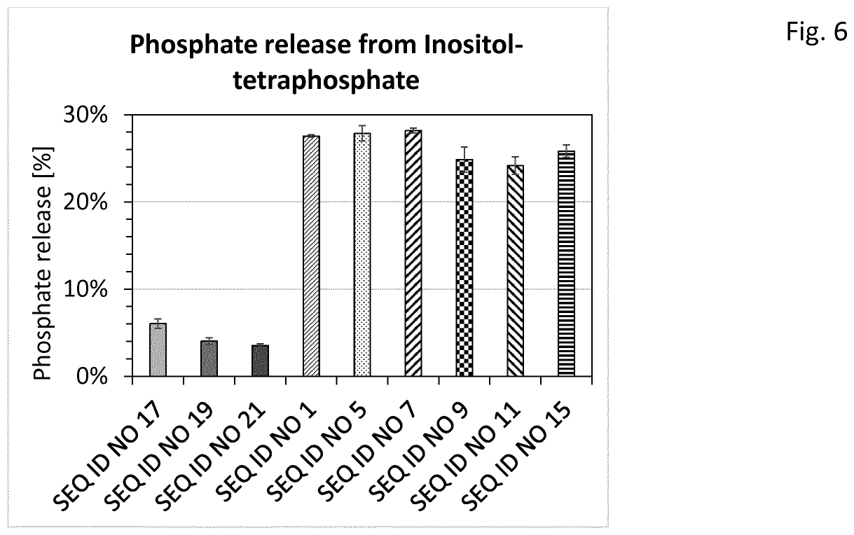

Results are shown in FIG. 6.

A sequence listing is provided as an ASCII text file named “2016DE604_adjusted_SequenceListing.txt” created on 7 Oct. 2019 and having a size of 61.4 kilobytes. The ASCII text file is hereby incorporated by reference in the application.

1. A method of preparing a feed or food product comprising adding at least one bacterial 3-phytase to an animal feed stuff to produce said feed or food product.

2. The method according to claim 1, wherein the phytase is characterized by less than 25% (wt./wt. free phosphate) accumulation of the intermediate product inositol-tetraphosphate after 35% inorganic phosphate release within an assay of 2.7 mmol/L phytate at 37° C. and pH 5.5 using an enzyme dosage of 0.2 U/mL.

3. The method according to claim 1, wherein the phytase is characterized by a phosphate release of at least 15% from 2.7 mmol/L phytate at pH 5.5 after 1 hour using an enzyme concentration of 0.21 μg/mL.

4. The method according to claim 1, wherein the phytase is a phytase from the genus Dickeya or Serratia.

5. The method according to claim 4, wherein the phytase is a phytase from Dickeya sp., Serratia sp., Dickeya zeae, Dickeya chrysanthemi, Dickeya dadantii, Dickeya solani or Dickeya paradisiaca.

6. The method according to claim 1, wherein the phytase shows at least 70% sequence identity to SEQ ID NO 1.

7. The method according to claim 1, wherein the phytase shows a temperature optimum of from 30 to 65° C. and/or a pH optimum of from pH 2.5 to 5.5.

8. The method according to claim 1, wherein the feed or food product further comprises at least one compound selected from the group consisting of vitamins, minerals, organic acids, probiotic components, oils, fats, pigments, growth factors, antimicrobial agents, cellulose, starch.

9. The method according to claim 1, wherein the feed or food product is in dry or liquid form.

10. A method of feeding an animal, comprising offering to the animal a feed or food product produced by the method of claim 1.

11. A method of preparing a feed or food additive comprising adding at least one bacterial 3-phytase to a composition to produce said food or feed additive.
